(19) United States
(12) Patent Application Publication
(10) Pub. No.: US 2009/0117076 A1
(43) Pub. Date: May 7, 2009

(54) Methods For Treating Tumor Cells

(76) Inventor: Lei L. Chen, Bountiful, UT (US)

Correspondence Address: THE MCCALLUM LAW FIRM, P. C., 685 BRIGGS STREET, PO BOX 929, ERIE, CO, 80516, US

(21) Appl. No.: 11/933,422

(22) Filed: Nov. 1, 2007

(51) Int. Cl. A61K 38/21; A61K 31/506; A61P 35/00; A61P 35/04

(52) U.S. Cl. 424/85.7; 514/252.18

(57) ABSTRACT

Methods of treating a disease in a patient are disclosed that include the administration of a targeted therapy in combination with an immunotherapy. Such therapy is useful in the treatment of any disease susceptible to targeted therapy and attack by the immune system.

### BACKGROUND

Treatments for cancer include chemotherapy, either alone, or in combination with other treatments, such as radiation or surgery. Chemotherapy is often administered as the combination of two or more agents in a variety of dosage regimens. Cancer cells are heterogenous and most can produce hormonal factors capable of paralyzing a patient's endogenous anti-tumor immunity.

Monotherapy using a targeted therapy alone will eliminate the drug-sensitive tumor cells, but not the tumor stem cells which are capable of renewal and proliferation whenever the targeted therapy is stopped. In addition, the effective targeted therapy provides a selective advantage for pre-existing drug-resistant clones, leading to the total dominance of the most aggressive drug-resistant clones, resulting in treatment failure, and ultimately death. The “tumor stem cells” and “drug-resistant clones” will remain the obstacles to cure for all cancer subtypes of cancer. For the minority of continued responders, the cancer will turn into a chronic disease and require the targeted therapy throughout the remainder of a patient's lifetime. For the majority of patients, the initial good response to targeted therapy will be short lived, followed by the emergence of drug-resistance and recurrence.

Numerous studies have identified combination therapies capable of achieving improved results when compared to the use of each of the respective treatments when used alone. However, in many cases the improved results are accompanied by adverse side effects that are difficult for patients to tolerate. Furthermore, some combination therapies ultimately suffer from the same or similar fates as the treatments when used alone, i.e., short lived responsiveness, drug-resistance, and recurrence of the disease.

Accordingly, there remains a need for combination therapies for the treatment of cancer that are minimally toxic with few adverse side effects at effective doses for achieving a significant clinical outcome, including long-term remission or cure.

The foregoing examples of the related art and limitations related therewith are intended to be illustrative and not exclusive. Other limitations of the related art will become apparent to those of skill in the art upon a reading of the specification.

### SUMMARY

In accordance with the purpose(s) of this invention, as embodied and broadly described herein, this invention, in one aspect, relates to a treatment for a disease including a first and second component, the first component being a quantity of a targeted therapy and the second component being a quantity of an immunotherapy where the treatment induces a immune response helpful in treating the disease.

In one embodiment, the targeted therapy is selected from the group consisting of tyrosine kinase inhibitors, antibodies and combinations of die same. In a particular embodiment, the tyrosine kinase inhibitor is selected from the group consisting of imatinib, sunitinib, sorafenib, nioltinib, lapatinib, gefitinib, erlotinib and dasatinib.

In another embodiment, the immunotherapy is selected from the group consisting of interleukins; an agent that stimulates said patient's own interferon production; dendritic cell therapy; tumor infiltrating lymphocyte therapy and combinations of the same.

In another embodiment, the targeted therapy is imatinib administered in an amount of between about 200 and 1000 mg/day and the immunotherapy is PEG-intron administered once per week during a first period in an amount of between about 0.5 and 6.0 mcg/kg and once per week in a second period in an amount of between about 0.5 and 2.0 mcg/kg.

In an alternate embodiment, the imatinib is administered in an amount of between about 300 and 900 mg/day and the immunotherapy is PEG-intron administered once per week during a first period in an amount of between about 1.0 and 5.0 mcg/kg and once per week in a second period in an amount of between about 0.75 and 1.5 mcg/kg.

In another embodiment, the imatinib is administered in an amount of between about 400 and 800 mg/day and the immunotherapy is PEG-intron administered once per week during a first period in an amount of between about 3.0 and 4.5 mcg/kg and once per week in a second period in an amount of between about 0.85 and 1.25 mcg/kg.

In an alternative embodiment, the imatinib is administered in an amount of about 400 mg/day and the immunotherapy is PEG-intron administered once per week during a first period in an amount of about 4.0 mcg/kg and once per week in a second period in an amount of about 1.0 mcg/kg.

In another embodiment, the imatinib is administered in an amount of between about 700 and 900 mg/day and the immunotherapy is PEG-intron administered once per week during a first period in an amount of between about 1.0 and 5.0 mcg/kg and once per week in a second period in an amount of between about 0.75 and 1.5 mcg/kg.

In an additional embodiment, the imatinib is administered in an amount of between about 700 and 900 mg/day and the immunotherapy is PEG-intron administered once per week during a first period in an amount of between about 3.0 and 4.5 mcg/kg and once per week in a second period in an amount of between about 0.85 and 1.25 mcg/kg.

In another embodiment, the imatinib is administered in an amount of between about 700 and 900 mg/day and the immunotherapy is PEG-intron administered once per week during a first period in an amount of about 4.0 and once per week in a second period in an amount of about 1.0 mcg/kg.

The first period of the present invention may be 7 weeks in duration and the second period may be 49 weeks in duration.

The PEG-intron of the present invention may be administered by subcutaneous injection.

In an embodiment, the PEG-intron is administered about 30 minutes following the administration of between about 500 to 1000 mg of acetaminophen.

In another embodiment, a CT scan at 24 weeks reveals that a patient has a surgically respectable disease and the patient is surgically rendered disease-free.

In a third embodiment, the disease is selected from the group consisting of cancer, and any condition, cancerous or otherwise, that might be susceptible to targeted therapy and attack by the immune system.

In a particular embodiment, the cancer is selected from the group consisting of a tumor, chronic myclogenous leukemia, and any cancer that might be susceptible to targeted therapy and attack by the immune system. In another particular embodiment, the tumor is gastrointestinal stromal tumors (“GIST”). In a third particular embodiment, the tumor exists in a patient that is KIT exon 11 negative.

### DETAILED DESCRIPTION

The present invention may be understood more readily by reference to the following detailed description of particular embodiments of the invention and Examples included therein.

Particular advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

Before the present invention and/or methods are disclosed and described, it is to be understood that this invention is not limited to specific compositions or treatment protocols, as such may, of course, vary, unless it is otherwise indicated. It is also to be understood that the terminology used herein is for the purpose of describing particular embodiments only and is not intended to be limiting.

### Definitions

For the purposes of the present invention, the following terms shall have the following meanings:

For the purposes of the present invention, the term “a” or “an” entity refers to one or more of that entity, for example, “a protein” or “an enzyme” refers to one or more of those elements or at least one element. As such, the terms “a” and “an”, “one or more” and “at least one” can be used interchangeably herein. It is also to be noted that the terms “comprising”, “including”, and “having” can be used interchangeably. Furthermore, “selected from the group consisting of” refers to one or more of the elements in the list that follows, including mixtures (i.e. combinations) of two or more of the elements.

Further more, for the purposes of the present invention, ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about”, it will be understood that the particular value forms another embodiment. It will be further understood that the endpoints of each of the ranges are significant both in relation to the other endpoint, and independently of the other endpoint.

Reference will now be made in detail to particular embodiments of the invention.

Two major obstacles of durable remission in cancer patients who demonstrate good response to targeted therapy or chemotherapy are acquired drug-resistant clones and tumor stem cells which can repopulate as soon as therapy is discontinued.

The present invention provides compositions and methods for a combination of effective targeted therapy and immunotherapy in order to treat a variety of diseases, including cancer.

Recognizing the problems with existing cancer therapies, the present invention provides compositions and methods for treatment aiming for cure (or long-term remission) including: (1) choosing an effective targeted therapy and a specific immunotherapy for each cancer subtype; and (2) designing a correct dose, timing and schedule of combining the targeted and immunotherapy for each cancer subtype. By correctly identifying the targeted therapy and the immunotherapy, it is possible to achieve cure or long-term remission by varying the timing, dosage, and through the described methods of monitoring treatment effects and analyzing the translational research. Combination of targeted therapy with specific immunotherapy, using proper dosages, and starting at the right time and schedules, has the potential to eradicate the drug-resistant clones and tumor stem cells upfront leading to cure or long-term remission.

Treatment of patients with an exemplary immunotherapy agent, imatinib mesylate (imatinib), a specific inhibitor of BCR-ABL, ABL, KIT, PDGFR-A, and PDGFR-B tyrosine kinases, for conditions such as chronic myelogenous leukaemia (CML), acute lymphoblastic leukemia, and gastrointestinal stromal tumor (GIST), can result in brisk and massive antigen release with minimal impairment of immune system. Unexpectedly, using a combination of effective targeted therapy and immunotherapy, it is possible to capture the massive antigen release from sensitive tumor cells, augment endogenous antitumor immunity against the pre-existing drug-resistant clones and tumor stem cells upfront, and achieve durable remission or cure.

The targeted therapy agents of the present invention may be any cancer therapeutic known in the air. Examples of suitable targeted therapy agents include, but are not limited to tyrosine kinase inhibitors, antibodies, combinations of the same and the like. Examples of suitable tyrosine kinase inhibitors include, but are not limited to imatinib, sunitinib, sorafenib, nioltinib, lapatinib, gefitinib, erlotinib, dasatinib and the like. All such targeted therapy agents can be administered alone or in combination with another.

In a particular embodiment, the targeted therapy agent is imatinib.

The immunotherapy of the present invention may be any agent capable of stimulating an immune response in a patient. Examples of suitable immunotherapy agents include, but are not limited to interleukins; agents that stimulate a patient's own interferon production; dendritic cell therapy; tumor infiltrating lymphocyte therapy, combinations of the same and the like. Exemplary interleukins may be selected from the group consisting of IL-2, toll-like receptor agonists, peptides, T-cell and B-cell receptor agonists, CpG-oligodeoxynucleotides, antibodies or antigenic fragments. In a particular embodiment, such immunotherapy agent is pegylated interferon α 2b (PEG-intron). An exemplary agent that is capable of stimulating a patient's own interferon production is imiquimod. In a particular embodiment, a patient's immune system may be stimulated by dendritic therapy whereby his or her own dendritic cells are harvested and stimulated to produce antigen prior to infusion back into the patient. In another particular embodiment, a patient's own tumor infiltrating lymphocytes can be harvested, expanded and reintroduced into the patient. All such immunotherapy agents can be administered alone or in combination with another agent.

An exemplary immunotherapy agent, Interferon α, through the intracellular mediators of the interferon-regulatory factor (IRF) family of transcription factors, is capable of optimizing the cytokine milieu by directing NK cell activation and augmenting its tumor lytic capacity, modulating antigen-presenting cells (APCs), modulating differentiation of T helper (T_{H}) cells, and developing effective adaptive antitumor immunity.

In a particular embodiment, the immunotherapy agent is PEG-intron. Such agent has increased plasma half-life and decreased clearance, and a once-weekly administration protocol, which eliminates the cyclical high and low plasma levels, leading to a much improved toxicity profile and tolerability than interferon-α 2b.

In one embodiment, the combination of targeted therapy and immunotherapy described herein is designed to: (1) induce apoptosis and necrosis of drug sensitive tumor cells by an effective targeted therapy; (2) restore endogenous anti-tumor immunity; (3) induce a shift from immune tolerance to development of robust innate (NK cells) and adaptive cytotoxicity anti-tumor immunity; and (4) eradicate the tumor stem cells and pre-existing drug-resistant clones upfront. With apoptosis and necrosis of most of sensitive tumor cells, the endogenous anti-tumor immunity can be restored, albeit weak and ineffective. Cytotoxic T lymphocytes (CTL) are capable of recognizing tumor specific antigens and kill tumor cells, but not normal cells. This can be achieved with the addition of immunotherapy at the right timing of massive release of tumor-specific antigens as a result of an effective targeted therapy. The empowered endogenous anti-tumor immunity can recognize and kill drug-sensitive, drug-resistant clones, and tumor stem cells without discrimination, and lead to cure or long-term remission.

In one embodiment, the targeted therapy is-Imatinib useful as a targeted therapeutic agent for GIST. It can induce brisk and massive tumor antigen release from rapid apoptosis and necrosis of imatinib-sensitive GISTs. Immunotherapy using PEG-intron has the potential to optimize the cytokine milieu, capture the massive release of tumor antigens, increase expression of class I MHC molecules, break the immune tolerance, promote innate immune response and enhance lytic potential of NK cells and adaptive antitumor immunity. This therapy protocol is designed to capture the unique features of both targeted therapy and immunotherapy to induce synergy in eradication of the drug-resistant clones as well as tumor stem cells upfront aiming for durable remission or cure.

The therapy of the present invention may be administered in any dose and/or dosing regime found to be effective in a patient. Such protocols are easily ascertained by one skilled in the art from the disclosures herein.

In one embodiment, the targeted therapy is imatinib administered in an amount of between about 200 and 1000 mg/day and the immunotherapy is PEG-intron administered once per week during a first period in an amount of between about 0.5 and 6.0 mcg/kg and once per week in a second period in an amount of between about 0.5 and 2.0 mcg/kg.

In an alternate embodiment, the imatinib is administered in an amount of between about 300 and 900 mg/day and the immunotherapy is PEG-intron administered once per week during a first period in an amount of between about 1.0 and 5.0 mcg/kg and once per week in a second period in an amount of between about 0.75 and 1.5 mcg/kg.

In another embodiment, the imatinib is administered in an amount of between about 400 and 800 mg/day and the immunotherapy is PEG-intron administered once per week during a first period in an amount of between about 3.0 and 4.5 mcg/kg and once per week in a second period in an amount of between about 0.85 and 1.25 mcg/kg.

In an alternative embodiment, the imatinib is administered in an amount of about 400 mg/day and the immunotherapy is PEG-intron administered once per week during a first period in an amount of about 4.0 mcg/kg and once per week in a second period in an amount of about 1.0 mcg/kg.

In another embodiment, the imatinib is administered in an amount of between about 700 and 900 mg/day and the immunotherapy is PEG-intron administered once per week during a first period in an amount of between about 1.0 and 5.0 mcg/kg and once per week in a second period in an amount of between about 0.75 and 1.5 mcg/kg.

In an additional embodiment, the imatinib is administered in an amount of between about 700 and 900 mg/day and the immunotherapy is PEG-intron administered once per week during a first period in an amount of between about 3.0 and 4.5 mcg/kg and once per week in a second period in an amount of between about 0.85 and 1.25 meg/kg.

In another embodiment, the imatinib is administered in an amount of between about 700 and 900 mg/day and the immunotherapy is PEG-intron administered once per week during a first period in an amount of about 4.0 and once per week in a second period in an amount of about 1.0 mcg/kg.

The first and second periods of the present invention may be any time necessary to achieve the desired therapeutic results. In a particular embodiment, the first period is 7 weeks in duration and the second period may be 49 weeks in duration. Such protocols are easily ascertained by one skilled in the art from the disclosures herein.

The targeted therapy and immunotherapy may be administered in any manner appropriate for the agent and patient. Such protocols are easily ascertained by one skilled in the art from the disclosures herein. In a particular embodiment, the PEG-intron of the present invention may be administered by subcutaneous injection.

In an embodiment, the PEG-intron is administered about 30 minutes following the administration of between about 500 to 1000 mg of acetaminophen.

In another embodiment, a CT scan at 24 weeks reveals that a patient has a surgically respectable disease and the patient is surgically rendered disease-free.

Treatment of any disease known in the art may be done by the compositions and methods of the present invention. Exemplary diseases include, but are not limited to cancer, any condition, cancerous or otherwise, that is susceptible to targeted therapy and attack by the immune system. Exemplary cancers include, but are not limited to gastrointestinal stromal tumors (“GIST”), chronic myelogenous leukemia, any cancer that might be susceptible to targeted therapy and attack by the immune system. In a third particular embodiment, the tumor exists in a patient that is KIT exon 11 negative. Such genetic test is well known to one skilled in the art.

### EXAMPLES

It should be appreciated by those skilled in the art that the techniques disclosed in the examples which follow represent techniques discovered by the inventors to function well in the practice of the invention, and thus can be considered to constitute particular modes for its practice. However, those of skill in the art should appreciate, in light of the present disclosure, that many changes can be made in the specific embodiments disclosed herein which will still obtain a like or similar result without departing from the spirit and scope of the invention.

### Therapeutic Agents

Imatinib is a small molecule selective inhibitor of tyrosine kinases ABL, BCR-ABL, KIT, PDGFRA, and PDGFRB, and is approved by FDA to treat GIST and several subtypes of hematological malignancies. Its relative molecular mass is 589.7 and its molecular formula is C₂₉H₃₁N₇O.CH₄SO₃.

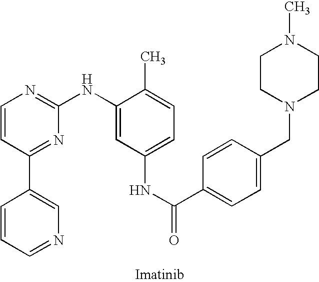

Commonly reported side effects to imatinib treatment included fluid retention, nausea, vomiting, diarrhea, muscle cramps, fatigue, and skin rash. Edema was most frequently periorbital or in lower extremities and adequately managed by diuretics. Severe superficial edema (CTC Grade ¾) was observed in 2% of GIST patients. Grade ¾ pleural effusion or ascites was observed in 2% of GIST patients. A recent finding of cardiac failure with an estimated incidence of 0.1% to 1.0% was reported in patients taking imatinib. In one embodiment, imatinib is adminisitered in a dose ranging between 200 and 1000 mg/day. In another embodiment, imatinib is administered in a dose ranging between 300 and 900 mg/day. In an alternate embodiment, imatinib is administered in a dose ranging between 400 and 800 mg/day. In another embodiment, imatinib is administered at a dose of 400 mg/day for patients where genotyping reveals a positive KIT exon 11 mutation and 800 mg/day for patients where genotyping reveals a negative KIT exon 11 mutation.

Most side effects are mild-to-moderate. Hematological, hepatic or renal toxicity are usually mild, dose-related, and are reversed by dose modification or discontinuation. Examples of medications that may increase the amount of imatinib in your blood include: ketoconazole, itraconazole, erythromycin, and clarithromycin. Examples of medications that may decrease the amount of imatinib in the blood include: dexamethasone, phenytoin, carbamazepine, rifampicin, Phenobarbital, and St. John's Wort.

Peglated Interferon α-2b (PEG-Intron) (also referred to as SCH 54031, PEG12000-interferon α-2b), is a covalent conjugate of recombinant interferon α-2b with monomethoxy polyethylene glycol (PEG, with an average molecular weight of 12,000 daltons). The conjugate is synthesized by reacting interferon α-2b with an electrophilic derivative of PEG. In one embodiment, PEG-intron is administered subcutaneously once per week for 7 weeks in doses in a range between 0.5 and 6.0 mcg/kg. In another embodiment, PEG-intron is administered at a high dose subcutaneously once per week for 7 weeks in doses in a range between about 1.0 and 5.0 mcg/kg. In an alternate embodiment, PEG-intron is administered subcutaneously once per week for 7 weeks in doses in a range between about 3.0 and 4.5 mcg/kg. In another embodiment, PEG-intron is administered subcutaneously once per week for 7 weeks at a dose of about 4.0 mcg/kg. In a preferred embodiment, after 7 weeks of the high dose treatment, PEG-intron is administered subcutaneously once per week for 49 weeks in doses ranging between about 0.5 and 2.0 mcg/kg. In another embodiment, after 7 weeks of the high dose treatment, PEG-intron is administered subcutaneously once per week for 49 weeks in doses ranging between about 0.75 and 1.5 mcg/kg. In an alternate embodiment, after 7 weeks of the high dose treatment, PEG-intron is administered subcutaneously once per week for 49 weeks in doses ranging between about 0.85 and 1.25 mcg/kg. In another embodiment, after 7 weeks of the high dose treatment, PEG-intron is administered subcutaneously once per week for 49 weeks in a dose of about 1.0 mcg/kg.

PEG-intron is a white to off-white lyophilized powder commonly supplied in 2-ml vials for subcutaneous use. Each vial contains either 74 mcg, 118.4 mcg, 177.6 mcg, or 222 mcg of PEG-intron, and 1.11 mcg dibasic sodium phosphate anhydrous, 1.11 mg monobasic sodium phosphate dihydrate, 59.2 mg sucrose, and 0.074 mg polysorbate 80. Following reconstitution with 0.7 ml of the supplied diluent (Sterile Water for Injection, USP), each vial contains PEG-intron at strengths of either 100 mcg/ml, 160 mcg/ml, 240 mcg/ml, or 300 mcg/ml.

The lyophilized powder is to be stored under refrigeration at 20° to 80° C. (360° to 460° F.). The reconstituted solution is stable between 20° C. and 80° C. (360° to 460° F.), and is to be used within 24 hours of reconstitution.

Two B-D Safety Lok syringes are provided in the package; one syringe is for the reconstitution steps and one for the patient injection. There is a plastic safety sleeve to be pulled over the needle after use. The syringe locks with an audible click when the green stripe on the safety sleeve covers the red stripe on the needle.

The PEG-intron lyophilized product is reconstituted with only 0.7 ml of supplied diluent (Sterile Water for injection, USP). The diluent vial is for single use only. The remaining diluent should be discarded. Swirl gently to hasten complete dissolution of the powder. The following equation is used to calculate the volume of each injection.

(1) PEG-Intron Dose of 4 mcg/kg:

$$Volume in mL = \frac{Weight ( kg ) \times Dose ( 4 mcg \text{/} kg )}{Reconstituted Vial Strength ( mcg \text{/} mL )} \\ PEG - intron Dose of 1 mcg \text{/} kg \text{:} \\ Volume in mL = \frac{Weight ( kg ) \times Dose ( 1 mcg \text{/} kg )}{Reconstituted Vial Strength ( mcg \text{/} mL )} \qquad ( 2 )$$

The appropriate PEG-intron dose should be withdrawn and injected subcutaneously. The PEG-intron vial is a single use vial and does not contain a preservative. Once the dose from a single dose vial has been opened, the sterility of any remaining product can no longer be guaranteed. The unused portion should be discarded.

The toxicity profile of PEG-intron in seven phase I studies is consistent with and quantitatively similar to that previously reported for interferon α-2b. The most common adverse events occurring with PEG-intron are flu-like symptoms, such as fever, fatigue, myalgia, headache, and nausea or vomiting, all of which usually decrease in severity as treatment continues. Some of these flu-like symptoms may be minimized by bedtime administration of the drug. Acetaminophen may be used to prevent or partially alleviate fever and headache. The majority of these symptoms was mild to moderate in severity, and did not appear to be dose-related. Application site disorder occurred frequently and included injection site inflammation, and reaction (i.e. bruise, itchiness, irritation, and/or pain). Decrease in platelet, white blood cell and neutrophil counts have been observed with PEG-intron administration in patients, all of which returned to normal upon discontinuation of treatment. Administration of PEG-intron was not associated with clinically significant changes in renal or liver function values in the majority of patients.

Since fever, rigor, nausea and other flu-like symptoms have been associated with PEG-intron, it should be used cautiously in patients with debilitating medical conditions, such as those with a history of cardiovascular disease, pulmonary disease, or diabetes mellitus. Caution should also be observed in patients with coagulation disorders or severe myelosuppression.

The carcinogenic effect of interferon a and the effect on fertility have not been studied, although interferon α-2b has been shown to be an abortifacient in primates.

### Example 1

This is a study of imatinib+PEG-intron for treatment of imatinib-naïve GIST patients. Initially all patients will be started with imatinib at 400 mg per day. If genotyping reveals negative KIT exon 11 mutation, the dose will be increased to 800 mg per day. Imatinib will be administered until progression. PEG-intron will be administered at 4 mcg/kg subcutaneously weekly for 7 weeks and then decreased to 1 mcg/kg subcutaneously weekly for 49 weeks thereafter.

If a patient has resectable disease by CT scan at 24 weeks, along with the surgeon's opinion that the patient can be surgically rendered disease-fee, the patient will undergo surgery at that time.

Response will be measured by both RECIST and Choi criteria (10% decrease in unidimensional tumor size or a 15% decrease in tumor density on contrast-enhanced CT scan). Although response achieved by targeted therapy is often swift, within 8 weeks, responses achieved with biological agents such as PEG-intron often are not seen until 24 weeks or longer after the start of therapy. Consequently, the traditional short-term response evaluation at 8 weeks is likely to miss a treatment advance. For this innovative combination of targeted and immunotherapy, the “time to response” is an important variable. Response will be evaluated at 8 weeks, 16 weeks, and 24 weeks, rather than at a single 8 week time point. The primary clinical outcomes include the time to response (including the response rate at each 8-week interval), progression and overall survival. These outcomes will be analyzed separately in the KIT exon 11 mutation positive and negative subgroups using the method of Kaplan and Meier and the Cox models accounting for KIT exon 11 mutational status and other prognostic variables. These data will be compared with historical clinical trial data using single agent imatinib.

For each patient, peripheral blood monocytes (PBMC) will be drawn by leukapheresis (50 ml blood) at baseline (pre-treatment), and 24 weeks (prior to surgery). Leukapheresis is optional at 3 weeks, 8 weeks, and at 2 and 4 years among the long-term continuous responders. This involves removing white blood cells and returning the plasma and remaining blood cells, including red blood cells and platelets.

For each sample, anti-tumor immunity by IFN-γ ELISPOT and immune modulatory effects of PEG-intron by NK & DC cell activation and cytokine profile will be evaluated. Based on an estimated 7 days to evaluate the laboratory study variables for each patient, a total of 7×30=210 laboratory days will be required; For each laboratory variable, univariate descriptive statistics will be computed at each time point and plots of the averages with suitable credible intervals over the time will be constructed.

### Treatment Plan

### Imatinib

Initially all patients will be started with imatinib at 400 mg per day. If the genotyping reveals negative KIT exon 11 mutation, the dose will be increased to 800 mg per day. For patients with BSA<1.54 m², the recommended imatinib dose is 260 mg/m²/day. Imatinib will be administered until progression.

### Dose Modification of Imatinib

### Hematological Adverse Reactions

The standard recommended imatinib dose is 400 mg/day for KIT exon 11 mutation positive patients, and 800 mg/day for KIT exon 11 mutation negative patients. Since imatinib is the active antitumor agent and is generally well tolerated, the imatinib dose will remain unchanged after genotypic analysis reveals the standard recommended dose. If patients suffer imatinib-related toxicities, they will be taken off of the protocol to receive an attenuated dose of imatinib.

TABLE 1
Hematological Toxicity Criteria for Imatinib Dose Modifications

| Dose Attenuation Criteria | Imatinib Dose |
|---|---|
| ANC ≦ 1.0 × 10⁹/L | 1. Stop imatinib until ANC > 1.5 × 10⁹/L |
| or | and platelet > 75 × 10⁹/L. |
| Platelet ≦ 50 × 10⁹/L | 2. Resume treatment with imatinib at |
| | the original recommended dose. |
| | 3. If recurrence of ANC ≦ 1.0 × 10⁹/L and or |
| | platelet ≦ 50 × 10⁹/L, repeat step 1, |
| | resume imatinib at full dose, and follow |
| | PEG-intron dose modification recommendation |
| | (section 7.4.1) |

### Hepatotoxicity and Other Non-Hematological Adverse Reactions

If severe non-hematological adverse reaction develops (such as severe hepatotoxicity or severe fluid retention or renal insufficiency), both imatinib and PEG-intron should be withheld until the event has resolved. Thereafter, treatment can be resumed at the original recommended dose.

### Peg-Intron

PEG-intron (Schering-Plough) will be administered at 4 mcg/kg by subcutaneous injection weekly for 7 weeks (Wk1 to Wk7), and then decreased to 1 mcg/kg subcutaneously weekly thereafter for 49 weeks (Wk8 to Wk56).

Patients can receive acetaminophen (500 mg to 1000 mg) 30 minutes prior to receiving PEG-intron if needed. Clinical nurses will administer the first dose in the outpatient clinic followed by observation in clinic for two hours after injection of this study medication. Subsequent doses of PEG-intron may be self-administered by the patient or his/her family member at home. Acetaminophen (500 mg to 1000 mg) may be used every 4-6 hours as needed for 2 days post each dose of PEG-intron.

### Dose Modification of PEG-Intron

### Hematological and Constitutional Adverse Reactions

TABLE 2
Toxicity Criteria for PEG-intron Dose Modifications

| Dose Attenuation Criteria | PEG-intron Dose |
|---|---|
| ANC ≦ 1.0 × 10⁹/L | 1. Stop PEG-intron until ANC > 1.5 × 10⁹/L or platelet > 75 × 10⁹/L |
| or | or reversal of constitutional symptoms from grade 3 to grade 1 |
| Platelet ≦ 50 × 10⁹/L | 2. Resume treatment with PEG-intron at the original starting dose of |
| or | 4 mcg/kg. |
| Grade 3 constitutional | 3. If recurrence of toxicity, repeat step 1 and resume PEG-intron at a |
| symptoms of fatigue, fever, | 25% reduction of the original dose (3 mcg/kg). |
| depression | 4. If 2^{nd} recurrence of toxicity at reduced dose, repeat step 1 and |
| or | resume PEG-intron at an additional 25% dose reduction (2.3 mcg/kg). |
| Grade 3 G1 symptoms of | 5. If 3^{rd} recurrence of toxicity at reduced dose, repeat step 1 and |
| nausea/vomiting, anorexia, | resume PEG-intron at an additional 25% dose reduction (1.7 mcg/kg). |
| | 6. If 4^{th} recurrence of toxicity at reduced dose, repeat step 1 and |
| | resume PEG-intron at 1 mcg/kg. |
| | 7. If 5^{th} recurrence of toxicity at PEG-intron dose of 1 mcg/kg, |
| | patient will be removed from protocol. |

### Hepatotoxicity and Other Non-Hematological Adverse Reactions

If severe non-hematological adverse reaction develops (such as severe hepatotoxicity or severe fluid retention or renal insufficiency), both imatinib and PEG-intron should be withheld until the event has resolved. Thereafter, treatment can be resumed at the original recommended dose. If the event takes more than 6 weeks to recover, or if the event recurs more than 4 times within 4 weeks, the patient will be taken off of the protocol. If grade 3 neuropsychiatric effects including depression, suicidal ideation, psychosis, hallucinations and aggressive behavior or GI symptoms or fatigue develops, dosage modification will be made as described in Table 2. Cardiac arrhythmias usually respond to conventional therapy but may require discontinuation of therapy.

### Evaluation for Surgery at Week 24

If the patient has resectable disease by CT scan at 24 weeks regardless of the response to treatment, along with the surgeon's opinion that the patient can be surgically rendered disease-free, the patient will undergo surgery at week 24-25. Surgery will be performed at a facility capable of harvesting fresh tumor cells for translational research. After surgery, patient should resume imatinib as soon as patient can tolerate a full meal. PEG-intron should be continued on schedule without interruption by surgery unless it is judged in appropriate by the treatment physician after discussion with PI.

### Evaluation During Treatment

### Interim History and Physical Examination

Physical examination, including assessments of body) weight and vital signs (blood pressure, heart rate, respiratory rate) as well as documentation of Karnofsky Performance Status will be performed.

Evaluation of toxicity and adverse events will be performed with each physical examination on each visit.

Complete blood count (CBC) with differential count, and comprehensive panel will be assessed upon each visit.

### Interim Evaluation of Target and Non-Target Lesions

Target and non-target legions are evaluated by CT scans at week 8, 16, and a pre-surgery evaluation at week 24. Then at week 32, 44, and 56, evaluations are repeated. Target and non-target legions are evaluated by PET-CT before treatment, at week 8, and week 24, are recommended as well.

### Duration of Treatment

All patients should continue therapy until study completion unless disease progression is documented on 2 successive occasions at least 4-8 weeks apart, or there is unacceptable toxicity.

### Criteria for Therapeutic Response Assessment

Target lesions (>2 cm) should be selected on the basis of their size (largest diameter) and their suitability for accurate repetitive measurement. A sum of the longest distance (LD) for all target lesions will be calculated and reported as the baseline sum LD. The baseline sum LD will be used as reference to further characterize the objective tumor response of the measurable dimension of the disease. Imaging-based evaluation is preferred to evaluation by clinical examination when both are available. Ultrasound may be useful for superficial palpable nodules.

All other lesions should be identified as non-target lesions and should also be recorded at baseline. Measurements are not required, but the presence or absence and qualitative description of each lesion should be recorded throughout follow-up.

All measurement should be recorded in metric notation using a ruler. All baseline evaluations should be performed as close to the treatment start as possible and never more than 4 weeks before the beginning of the treatment. CT and/or PET-CT will be required at weeks 8, 16 and at pre-surgery evaluation at week 24.

### Clinical Complete Response (CR)

A complete response will be determined by the disappearance of all evidence of tumor for at least four weeks. The patient must be free of all symptoms of cancer.

### Pathologic Complete Response (PCR)

A pathologic complete response will be identified by a complete lack of evidence of viable tumor in the surgical resected specimen of all target and non-target lesions.

### Partial Response (PR)

Partial responses to the described treatment methodology will be determined as follows:

RECIST A 30% decrease in the sum of the longest diameter (LD) of target lesions taking as reference the baseline sum LD persisting for at least 4 weeks.

Choi criteria: 10% decrease in unidimensional tumor size OR a 15% decrease in tumor density (HU) on contrast-enihanced CT scan. No obvious progression of non-measurable disease.

### Progression of Disease (PD)

Progression of disease will be determined as follows:

RECIST: At least a 20% increase in the sum of the longest diameter (LD) of target lesions, taking as reference the baseline sum LD recorded since the start of treatment. The appearance of new lesions and/or unequivocal progression of existing non-target lesions are also considered progression.

Choi criteria: An increase in unidimensional tumor size of ≧10% AND did not meet CT scan density criteria for PR (HU decrease by 15%). Any new lesions, including new tumor nodules in a previous cystic tumor.

### Stable Disease (SD)

A stable disease state will be characterized by either sufficient shrinkage to qualify for partial response nor sufficient increase to qualify for progressive disease, with no symptomatic deterioration.

### Confirmation

To be assigned a status of PR or CR or Progression, changes in tumor measurements must be confirmed by repeat studies that are performed 4-8 week after criteria for response are first met.

### Duration of Response

Duration of response is measured from the time measurement criteria are met for CR or PR until the first date that progression is objectively documented.

### Statistical Considerations

### Treatment and Study Rationale

Initially all patients will be started at imatinib 400 mg per day, as soon as the genotyping reveals negative KIT exon 11 mutation, the dose will be increased to 800 mg per day. Imatinib will be administered until progression. PEG-intron will be administered at 4 mcg/kg subcutaneously weekly×7 weeks, and then decreased to 1 mcg/kg subcutaneously weekly thereafter for 49 weeks. Each patient's tumor will be evaluated by CT scan at 8 weeks, 16 weeks, and 24 weeks and every 3 months until progression. PET-CT scan at various time points of treatment of GIST are critically important in judging response to therapy, and minimally 3 PET-CT scan are recommended in this study, pre-treatment, 8 weeks, and at time of progression. Based on these evaluations and with the surgeon's opinion that the patient can be surgically rendered disease-free, the patient will undergo surgery at 24 weeks.

### Outcomes

Both clinical and immunological outcomes will be monitored.

### Efficacy

Response will be measured by both RECIST and Choi criteria (10% decrease in unidimensional tumor size or a 15% decrease in tumor density on contrast-enhaniced CT scan). Although response achieved by targeted therapy is often swift, within 8 weeks, responses achieved with biological agents such as PEG-intron often are not seen until 24 weeks or longer after the start of therapy. Consequently, the traditional short-term response evaluation at 8 weeks is likely to miss a treatment advance. For this innovative combination targeted and immunotherapy, the “time to response” is an important variable. Response will be evaluated at 8 weeks, 16 weeks, and 24 weeks, rather than at a fixed 8 weeks time point. The primary clinical outcomes include the times to response (including the response rate by each 8-week interval), time to progression and overall survival. These data will be compared with historical clinical trial data using single agent imatinib. A doubling of the median time to progression as compared to the historical values will be considered efficacious.

### Laboratory Studies

For each patient, peripheral blood monocytes (PBMCs) will be drawn by leukapheresis at baseline (pre-treatment), weeks 3, and 24 (prior to surgery within 7 days), 8 weeks and at 2 and 4 years are optional among the long-term continuous responders. For each sample, the following variables will be assessed by, laboratory studies: Anti-tumor immunity by IFN-γ ELISPOT; all PBMC and primary tumor cells prepared from the unused fresh tumors will be cryopreserved; and autologous viable tumor cells are required for demonstration of anti-tumor immunity by IFN-γ ELISPOT. Preferably, fresh viable tumor is available at the time of surgery. IFN-γ ELISPOT cannot be performed if there is complete tumor response (no viable tumor cells left) or tumor progression (no surgery). Immunomodulatory effect of PEG-intron will be determined by assessing NK & DC cell activation and serum cytokine profiles.

### Example 2

Treatment procedures, study details, and analysis for Example 2 are the same as described previously for Example 1, but also include the following.

### Sample Size and Interim Monitoring

A maximum of 30 patients will be treated. The anticipated accrual rate is 15 to 30 patients per year, so including a minimum follow up of 3 years after completion of PEG-intron treatment of the last patient, maximum trial duration of 6 years is anticipated. With this sample size and recruitment/follow-up time, the study has an at least 78% power to detect a doubling of the median time to progression as compared to the historical values of 687 days and 200 days for the two subgroups with and without KIT exon 11 mutation.

### Resources

Based on an estimated 7 days to evaluate the laboratory study variables for each patient, a total of 7×30=210 laboratory days will be required.

### Data Analyses

The distributions of the time to response, response rate, overall survival time, and time to progression among responders, each will be estimated separately in the KIT exon 11 mutation positive and negative subgroups using the method of Kaplan and Meier (E. L. Kaplan & P. Meier, Nonparametric Estimator from Incomplete Observations, 53 J. Am. Statistical Assoc. 457-81 (1958)), incorporated hererin by reference, and also using Cox models (D. R. Cox, Regression Models and Life Tables, 34 J. R. Statistical Soc. 187-220 (1972)), incorporated herein by reference, accounting for KIT exon 11 mutational status and other prognostic variables. For each laboratory variable, univariate descriptive statistics will be computed at each time point (baseline, 3 week, 6 week, 24 week, 2 ),ears, and optional at 4 years), and plots of the averages with suitable credible intervals over the time will be constructed.

### Example 3

### Translational Research and Immune Monitory Analysis

Treatment procedures, study details, and analysis for Example 3 are the same as described previously for Examples 1 and 2, but also include the following.

Genotyping

A paraffin block or 7 unstained slides are required prior to enrollment. DNA will be extracted from the paraffin embedded GIST for sequencing analysis of KIT and PDGFRA for all patients prior to or immediately after treatment.

Primary Tumor Cell Preparation

Unused surgical specimens will be collected fresh under sterile conditions for primary tumor cell preparation, and will be cryopreserved for later antitumor immunity (IFN-γ ELISPOT assay), intratumoral analysis, and molecular biological studies.

Immune Modulatory Effects of Peg-Intron

At each time point of leukapheresis, PBMC and 4 ml of serum will be cryopreserved for later use in the following assays.

Immune Modulation on NK Cells

Immune modulation on NK cells will be assessed by isolation of NK cells by positive or negative selection and assessment of NK cell activation by IFN-γ secretion.

Immune Modulation on APCs

Immune modulation on APCs will be assessed by isolation of DCs by brief adherence and assessment of DC activation by IL-12 secretion.

Leukocyte phenotype analysis will be assessed by flow cytometry. Analysis of CD4+ and CD8+ T-lymphocyte subsets, status of 45RA and 45RO in CD4+ and CD8+ T-lymphocytes, CD4+CD8+FOXP3+T_{reg} cells, and CD56+NK cells will be tested.

Serum Cytokine Profile Analysis

Serum cytokine profile analysis will be accomplished by luminex multiplex testing of a panel of relevant cytokines including: IFN-γ, IL-1b, IL-2, IL-2 receptor, IL-4, IL-5, IL-6, IL-8, IL-10, IL-12 p70, IL- 13, TNF-α, IFN-γ, CD40 ligand. ELISA testing of a panel of relevant cytokines will include the following: IL-15, IL-17, CCR4, CXCR3, CCR5, TGF-β, and INF-γ.

Antitumor Response

Antitumor response will be assessed from the patient's autologous primary tumor cells that were harvested and cryopreserved from unused fresh surgical specimens and will be used to stimulate T-lymphocytes, and serve as target cells for the tumor-specific CTL analysis by IFN-γ ELISPOT assay.

### Example 4

### Biomarker Studies

Treatment procedures, study details, and analysis for Example 4 are the same as described previously for Examples 1, 2, and 3, but also include the following.

Proteomic analysis will be accomplished through the use of combined chromatography and mass spectrometry by the Surface Enhanced Laser Desorption/Ionization-time of Flight Mass Spectrometry (SELDI-TOF MS). This proteomic study has the potential of providing meaningful information because of the following 3 unique features of this clinical trial: (i) imatinib is an effective targeted therapy demonstrating 69% overall response rate of tumor necrosis/apoptosis, and exhibiting minimal undue toxicity to normal cells; (ii) the comparison is made at different time point of treatment within the same patient, thus reduces individual variability and background noise; (iii) direct correlation of proteomic data with clinical response and parallel laboratory studies.

The compositions and methods disclosed and claimed herein can be made and executed without undue experimentation in light of the present disclosure. While the compositions and methods of this invention have been described in terms of particular embodiments, it will be apparent to those of skill in the art that variations may be applied to the compositions and methods and/or in the steps or in the sequence of the methods described herein without departing from the concept, spirit and scope of the invention. More specifically, it will be apparent that certain related reagents may be substituted for the reagents described herein while the same or similar results would be achieved. All such substitutes and modifications apparent to those skilled in the art are deemed to be within the spirit, scope and concept of the invention as defined by the appended claims.

We claim:

1. A method of treating a disease, comprising;
administering to a patient a treatment comprising, an amount of a first component and an amount of a second component, said first component being a quantity of a targeted therapy, said second component being a quantity of an immunotherapy, wherein said treatment induces a immune response helpful in treating said disease.

2. The method of claim 1, wherein said targeted therapy is selected from the group consisting of tyrosine kinase inhibitors, antibodies and combinations of the same.

3. The method of claim 2, wherein said tyrosine kinase inhibitor is selected from the group consisting of imatinib, sunitinib, sorafenib, nioltinib, lapatinib, gefitinib, erlotinib and dasatinib.

4. The method of claim 1, wherein said immunotherapy is selected from the group consisting of interleukins; an agent that stimulates said patient's own interferon production; dendritic cell therapy; tumor infiltrating lymphocyte therapy and combinations of the same.

5. The method of claim 1, wherein said targeted therapy is imatinib administered in an amount of between about 200 and 1000 mg/day and the immunotherapy is PEG-intron administered once per week during a first period in an amount of between about 0.5 and 6.0 mcg/kg and once per week in a second period in an amount of between about 0.5 and 2.0 mcg/kg.

6. The method of claim 5, wherein imatinib is administered in an amount of between about 300 and 900 mg/day and the immunotherapy is PEG-intron administered once per week during a first period in an amount of between about 1.0 and 5.0 mcg/kg and once per week in a second period in an amount of between about 0.75 and 1.5 meg/kg.

7. The method of claim 6, wherein imatinib is administered in an amount of between about 400 and 800 mg/day and the immunotherapy is PEG-intron administered once per week during a first period in an amount of between about 3.0 and 4.5 mcg/kg and once per week in a second period in an amount of between about 0.85 and 1.25 mcg/kg.

8. The method of claim 7, wherein imatinib is administered in an amount of about 400 mg/day and the immunotherapy is PEG-intron administered once per week during a first period in an amount of about 4.0 mcg/kg and once per week in a second period in an amount of about 1.0 mcg/kg.

9. The method of claim 8, wherein said first period is 7 weeks in duration and said second period is 49 weeks in duration.

10. The method of claim 9, wherein the PEG-intron is administered by subcutaneous injection.

11. The method of claim 10, wherein said treatment of PEG-intron is administered about 30 minutes following the administration of between about 500 to 1000 mg of acetaminophen.

12. The method of claim 10, wherein a CT scan at 24 weeks reveals that a patient has a surgically respectable disease and the patient is surgically rendered disease-free.

13. The method of claim 1, wherein said disease is selected from the group consisting of cancer and any disease susceptible to targeted therapy and attack by the immune system.

14. The method of claim 13, wherein said cancer is selected from the group consisting of a tumor, chronic myelogenous leukemia, and any cancel that might be susceptible to targeted therapy and attack by the immune system.

15. The method of claim 14, wherein said tumor is gastrointestinal stromal tumors (“GIST”).

16. The method of claim 14, wherein said tumor exists in a patient that is KIT exon 11 negative.

17. The method of claim 7, wherein said imatinib is administered in an amount of between about 700 and 900 mg/day and the immunotherapy is PEG-intron administered once per week during a first period in an amount of between about 1.0 and 5.0 mcg/kg and once per week in a second period in an amount of between about 0.75 and 1.5 mcg/kg.

18. The method of claim 15, wherein said imatinib is administered in an amount of between about 700 and 900 mg/day and the immunotherapy is PEG-intron administered once per week during a first period in an amount of between about 3.0 and 4.5 mcg/kg and once per week in a second period in an amount of between about 0.85 and 1.25 mcg/kg.

19. The method of claim 18, wherein said imatinib is administered in an amount of between about 700 and 900 mg/day and the immunotherapy is PEG-intron administered once per week during a first period in an amount of about 4.0 and once per week in a second period in an amount of about 1.0 mcg/kg.

20. The method of claim 17, wherein said treatment of PEG-intron is administered about 30 minutes following the administration of between about 500 to 1000 mg of acetaminophen.
